(19) United States
(12) Patent Application Publication
(10) Pub. No.: US 2024/0148602 A1
(43) Pub. Date: May 9, 2024

(54) VACUUM MANIFOLD FOR STRETCHING PROXIMAL CORPORA OF A USER

(71) Applicant: Benjamin Deane Sadler, Denver (CO)

(72) Inventor: Benjamin Deane Sadler, Denver (CO)

(21) Appl. No.: 18/386,966

(22) Filed: Nov. 3, 2023

Related U.S. Application Data
(60) Provisional application No. 63/422,829, filed on Nov. 4, 2022.

(51) Int. Cl. A61H 19/00; A61H 9/00

(52) U.S. Cl. CPC A61H 19/32; A61H 9/0057

(57) ABSTRACT

A vacuum manifold for stretching proximal corpora of a user, including a body, an inner chamber, a vacuum attachment, a first opening, and a second opening. The body extends from a proximal end to a distal end, and from a top end to a bottom end. The inner chamber is defined by the body. The vacuum attachment is configured to provide fluid communication from a vacuum source to the inner chamber. The first opening receives a perineum of the user into the inner chamber and is located at the top end of the body. The second opening receives a penile shaft of the user and extends from the inner chamber to the distal end. The inner chamber of the vacuum manifold is configured to apply pressure on a proximal corpora of the user such that the proximal corpora is stretched radially and azimuthally.

### CROSS-REFERENCE TO RELATED APPLICATIONS

This application claims priority to U.S. Provisional Application No. 63/422,829, filed Nov. 4, 2022, the entire contents of which is hereby incorporated by reference.

### TECHNICAL FIELD

The present disclosure relates generally to vacuum erection devices, and in particular, relates to a vacuum manifold for stretching proximal corpora of the user.

### BACKGROUND

Vacuum therapy can be used to stretch penile tissues for decreasing pain in the penis due to injury and/or for relieving sexual dysfunction. Vacuum erection devices (VEDs) are mechanical means for stretching the penile tissues. For example, VEDs generate a negative pressure around the penis to stretch the penile tissues. A distal portion of the penile shaft is inserted into the VED and the tissues of the distal portion of the penile shaft are stretched both width-wise and length-wise. The stretching of the distal portion causes a proximal portion (e.g., a portion of the penile shaft located internally) to stretch length-wise. Such VEDs, however, are unable to stretch the proximal portion of the penile shaft width-wise. Therefore, there is a need for a device that stretches the proximal portion of the penile shaft width-wise for providing therapy to the proximal portion.

### BRIEF DESCRIPTION OF THE DRAWINGS

The foregoing and other features and advantages will be apparent from the following, more particular, description of various exemplary embodiments, as illustrated in the accompanying drawings, wherein like reference numbers generally indicate identical, functionally similar, and/or structurally similar elements.

FIG. 1A is a bottom, inferior view of a penis of a user, according to the present disclosure.

FIG. 1B is a side view of the penis of the user of FIG. 1A, according to the present disclosure.

FIG. 1C is a conceptual representation of a perspective front side view of the penis of the user of FIGS. 1A and 1B, according to the present disclosure.

FIG. 1D is a schematic, cross-section view of the penis of the user, taken at line 1D-1D in FIG. 1B, according to the present disclosure,

FIG. 2 is a conceptual representation of a schematic, cutaway side view of a user in an area of the penis of FIGS. 1A to 1D with an existing vacuum erection device (VED), according to the present disclosure.

FIG. 3A is a bottom, inferior view of the penis of the user of FIGS. 1A to 1D with a vacuum manifold applied to the user, according to the present disclosure.

FIG. 3B is a conceptual representation of a schematic, cutaway side view of the user in an area of the penis of FIGS. 1A to 1D with the vacuum manifold of FIG. 3A applied to the user, according to the present disclosure.

FIG. 4 is a perspective view of the vacuum manifold of FIGS. 3A and 3B isolated from a vacuum erection device, according to the present disclosure.

FIG. 5 is a perspective side view of a system for stretching proximal corpora of a user including the vacuum manifold of FIG. 4 attached to a vacuum erection device, according to the present disclosure.

FIG. 6 is a perspective view of a part of a vacuum manifold, according to another embodiment.

FIG. 7 is a perspective side view of a system for stretching proximal corpora of a user including the vacuum manifold of FIG. 6 attached to a vacuum source, according to another embodiment.

FIG. 8 is a flow diagram of a method of stretching proximal corpora of a user, according to the present disclosure.

### DETAILED DESCRIPTION

Additional features, advantages, and embodiments of the present disclosure are set forth or apparent from a consideration of the following detailed description, drawings, and claims. Moreover, both the foregoing summary of the present disclosure and the following detailed description are exemplary and intended to provide further explanation without limiting the scope of the disclosure as claimed.

Various embodiments of the present disclosure are discussed in detail below. While specific embodiments are discussed, this is done for illustration purposes only. A person skilled in the relevant art will recognize that other components and configurations may be used without departing from the spirit and the scope of the present disclosure.

As used herein, the terms “first” and “second” may be used interchangeably to distinguish one component from another and are not intended to signify location or importance of the individual components.

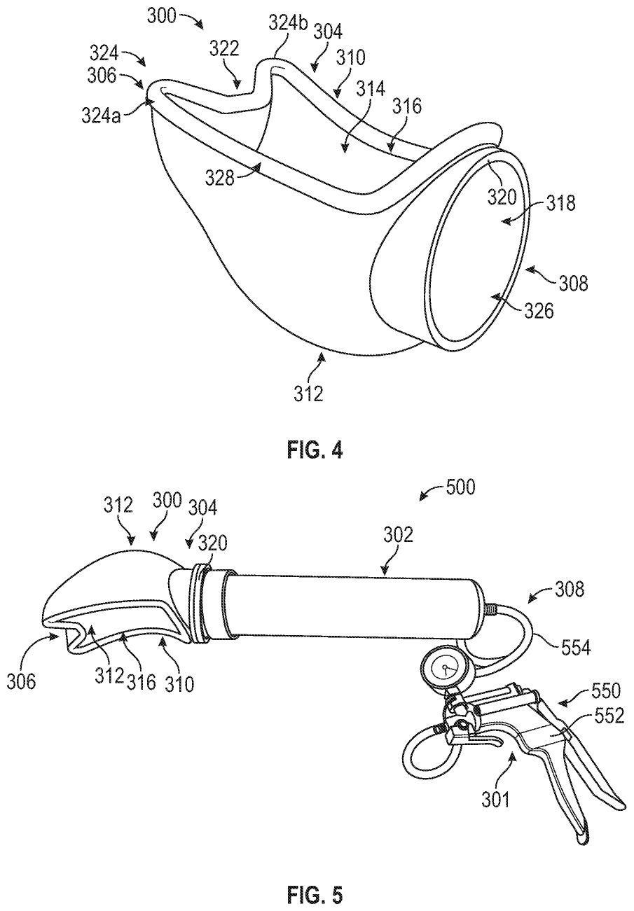

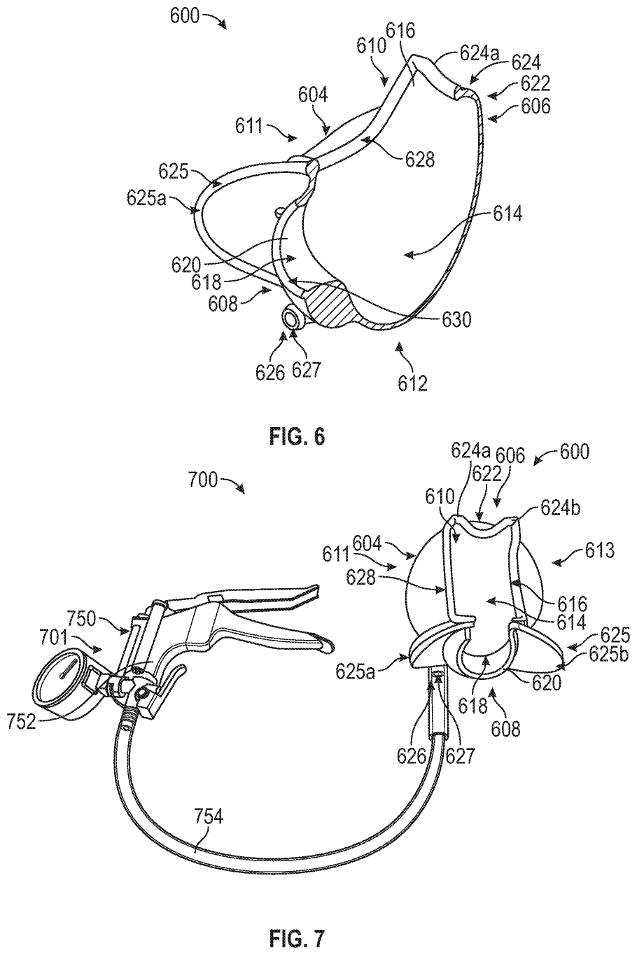

The terms “front,” “rear,” “top,” “bottom,” “right side,” and “left side” refer to relative positions of the vacuum manifold based on the orientation shown in FIGS. 4 and 6, respectively, unless stated otherwise.

The terms “coupled,” “fixed,” “attached,” “connected,” and the like, refer to both direct coupling, fixing, attaching, or connecting, as well as indirect coupling, fixing, attaching, or connecting through one or more intermediate components or features, unless otherwise specified herein.

The singular forms “a,” “an,” and “the” include plural references unless the context clearly dictates otherwise.

As used herein, the terms “axial” and “axially” refer to directions and orientations that extend substantially parallel to a centerline of a component. Moreover, the terms “radial” and “radially” refer to directions and orientations that extend substantially perpendicular to the centerline of the component. In addition, as used herein, the terms “circumferential” and “circumferentially” refer to directions and orientations that extend arcuately about the centerline of the component.

Approximating language, as used herein throughout the specification and claims, is applied to modify any quantitative representation that could permissibly vary without resulting in a change in the basic function to which it is related. Accordingly, a value modified by a term or terms, such as “about,” “approximately,” “generally,” and “substantially” is not to be limited to the precise value specified. In at least some instances, the approximating language may correspond to the precision of an instrument for measuring the value, or the precision of the methods or the machines for constructing the components and/or the systems or manufacturing the components and/or the systems. For example, the approximating language may refer to being within a one, two, four, ten, fifteen, or twenty percent margin in either individual values, range(s) of values and/or endpoints defining range(s) of values.

Penile fibrosis is a condition caused by physical or chemical injury. Penile fibrosis is the existence of scar tissue inside the penis. When the delicate internal penile tissues are replaced with tough scar tissue, patients may experience sexual dysfunctions, pain, a narrowed (stenosis) or shortened penis, and a greatly diminished quality of life. Stretching provides general benefits to the healing process of injured tissue, and is an effective treatment for penile fibrosis and Peyronie's disease. Vacuum therapy is one method to stretch penile tissues, and decreases pain and sexual dysfunction associated with penile fibrosis and vascular disorders through suspected vasoactive exercise of erectile tissue. Current extant vacuum therapy devices, however, cannot radially or azimuthally stretch the tissues of the proximal corpora—such devices can only stretch the tissues or the proximal corpora axially. The penile corpora extend from the tip of the glans, though the penile shaft and scrotum, and then diverge within the perineum and terminate on either side of the anus. Current extant vacuum erection devices (VEDs) apply vacuum pressure only to the distal portion of the penis (shaft and glans). Some VEDs, referred to as “combination” penis pumps, include applying pressure to the scrotum.

The deep fascia is a cylindrical sheath of connective tissue surrounding the corpora. The deep fascia is an inelastic structural pressure vessel. The tunica albuginea is also a cylindrical sheath but is more elastic than the fascia. The tunica albuginea fills with blood during an erection. The corporeal trabeculae are small, helical blood vessels that radiate outwards from the central arteries of each corpus and terminate at the inner tunical walls. The corporeal trabeculae are tiny filaments that comprise an interior “sponge” of vessels in healthy penises. Due to their geometry, the deep fascia, the tunica albuginea, and the corporeal trabeculae can only be stretched in certain dimensions. For example, the deep fascia and the tunica albuginea can only be stretched axially and azimuthally, but not radially. The corporeal trabeculae can be stretched axially, radially, and azimuthally.

Traditional VEDs use a cylindrical vacuum manifold to establish a 15-30 kPa negative pressure outside the distal shaft of the penis. The negative pressure substantially increases the intracorporeal pressure to approximately double or more, in the distal portion only, stretching the tissues of the distal penis in all three cylindrical dimensions (e.g., axial, radial, and azimuthal). However, the proximal portions of the corpora are left out of the vacuum manifold, and remain unpressurized due to other tissues (e.g., mostly skin from the pubic symphysis and scrotum) being suctioned into the open base end of the VED, thereby creating an annular “plug” of flesh around the base of the penis, which seals the vacuum roughly halfway along the length of the corpora. Axial tension is transmitted through the proximal corpora due to the distal corpora being anchored to the body through the proximal corpora, though the proximal corpora remain unpressurized. Further, after the distal shaft has been pressurized, a flexible ring can be applied to the base of the penis to keep the distal part pressurized after the VED has been removed. While current VEDs apply axial stress to the proximal corpora, a proximal fibrotic stenosis requires applying radial traction to the proximal trabeculae, and azimuthal traction (e.g., hoop stress) to the proximal tunica and deep fascia.

The vacuum manifolds of the present disclosure create a pressure distribution which is capable of stretching the proximal tissues of the penis in the radial and azimuthal dimensions. For example, the vacuum manifolds are configured to apply vacuum pressure to the skin superficial to the full length of the corpora, thereby achieving full radial and azimuthal stretching of the proximal corpora. The vacuum manifolds are configured to apply pressure to the proximal corpora to stretch the proximal corpora. Stretching the proximal corpora in such a way is different than current VEDs that carry axial tension to stretch the proximal corpora only axially. Stretching the proximal corpora in such a way provides for treatment of a proximal fibrotic stenosis or other injuries in the area of the proximal corpora. Further, stretching the proximal corpora in such a way provides for enhanced sexual stimulation. For example, the vacuum manifolds herein can be utilized to pressurize the proximal corpora for sexual stimulation. Thus, the vacuum manifolds of the present disclosure provide for therapeutic benefits as well as non-therapeutic benefits.

The present disclosure includes various embodiments of the vacuum manifold. In one embodiment, the vacuum manifold is used in combination with a current VED and extends the treatment region from the distal penis to include the proximal corpora. In another embodiment, the vacuum manifold is a stand-alone device that applies a vacuum only to the proximal corpora and excludes the distal penis by means of a seal around the proximal shaft of the penis. The vacuum manifolds disclosed herein is configured to apply vacuum pressure to the skin directly superficial to the deep proximal corpora, while excluding sensitive adjacent structures (e.g., testes, spermatic cords, inguinal canals, and anus) from experiencing the vacuum pressure. This is achieved by the following features of the vacuum manifolds: (a) a bifurcated posterior (in order to pressurize the diverging proximal corpora without pressurizing the anus), (b) a narrow anterior opening and wings to secure the testes and scrotal skin outside of the vacuum area, and (c) a general morphology that allows an air-tight fit around the anterior male perineum. Additional features of the vacuum manifold of the present disclosure are detailed below.

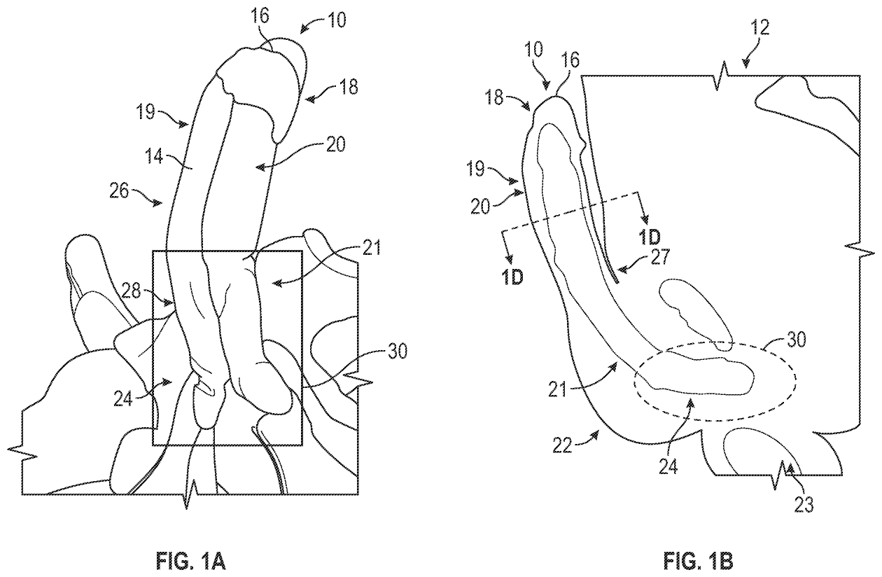

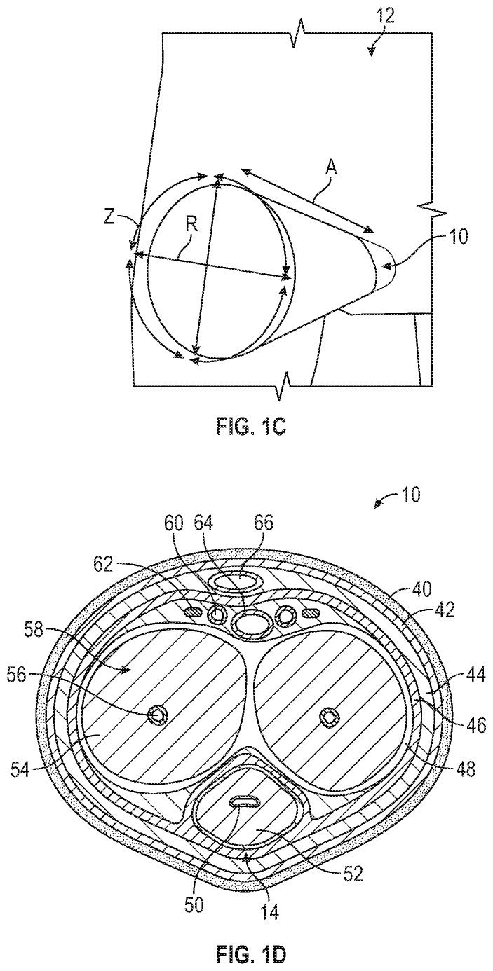

Referring now to the drawings, FIG. 1A is a bottom, inferior view of a penis 10 of a user 12, according to the present disclosure. FIG. 1B is a side view of the penis 10 of the user 12, according to the present disclosure. FIGS. 1A and 1B show the penile corpora 14 of the penis 10. The penile corpora 14 extends from a tip 16 of the glans 18, through a penile shaft 20 and a scrotum 22 (including testicles 23), and diverges within a perineum 24. The penile shaft 20 includes a distal portion 19 and a proximal portion 21. The distal portion 19 of the penile shaft 20 is the external portion of the penile shaft 20 that extends from the user's body (e.g., from the pubic symphysis 27). The proximal portion 21 of the penile shaft 20 is the internal portion of the penile shaft 20 that extends internally. The portion of the penile corpora 14 from the tip 16 to a pubic symphysis 27 of the user 12 is referred to as a distal corpora 26. The distal corpora 26 is the portion of the penis 10 that is external to the user's body and extends from the pubic symphysis 27 of the user 12. The portion of the penile corpora 14 that diverges within the perineum 24 is referred to as a proximal corpora 28. In particular, FIGS. 1A and 1B show an injury area 30 to the proximal corpora 28. The proximal corpora 28 can become injured due to physical or chemical injury, as detailed above.

FIG. 1C is a conceptual representation of a perspective front side view of the penis 10, according to the present disclosure. FIG. 1C shows the penis 10, and the components thereof, can be defined using a cylindrical coordinate system. For example, the cylindrical coordinate system includes an axial direction A, a radial direction R, and an azimuthal direction Z. The axial direction A is defined horizontally along a length of the penis 10, the radial direction R is defined from a center of the penis 10 laterally outwards, and the azimuthal direction Z is defined by a circular path (e.g., at a particular radius) about the center of the penis 10. For example, the azimuthal direction Z is defined circumferentially about the penis 10.

FIG. 1D is a schematic, cross-sectional view of the penis 10, taken at line 1D-1D in FIG. 1B, according to the present disclosure. FIG. 1D shows the penis 10 includes penile skin 40, superficial fascia 42, areolar tissue 44, deep fascia 46, tunica albuginea 48, a urethra 50, corpus spongiosum 52, corpus cavernosum 54, deep arteries 56, corporeal trabeculae 58, dorsal arteries 60, deep dorsal nerves 62, a deep dorsal vein 64, and a superficial dorsal vein 66. Together, the corpus spongiosum 52 and the corpus cavernosum 54 form the penile corpora 14 of the penis 10. Of particular importance are the deep fascia 46, the tunica albuginea 48, and the corporeal trabeculae 58. The deep fascia 46 is a cylindrical sheath of connective tissue surrounding the penile corpora 14. The deep fascia 46 is an inelastic structural pressure vessel. The tunica albuginea 48 is a cylindrical sheath and is more elastic than the deep fascia 46. The tunica albuginea 48 fills with blood during an erection. The corporeal trabeculae 58 are small, helical blood vessels that radiate outwards from the central arteries of each corpus (e.g., the corpus spongiosum 52 and the corpus cavernosum 54) and terminate at the inner tunical walls. The corporeal trabeculae 58 are tiny filaments that comprise an interior sponge of vessels in healthy penises.

The deep fascia 46, the tunica albuginea 48, and the corporeal trabeculae 58 can only be stretched in certain dimensions due to geometries of the deep fascia 46, the tunica albuginea 48, and the corporeal trabeculae 58. With reference to FIG. 1C, the deep fascia 46 and the tunica albuginea 48 can each be stretched in the axial direction A and in the azimuthal direction Z, but not in the radial direction R. The corporeal trabeculae 58 can be stretched in the axial direction A, in the radial direction R, and in the azimuthal direction Z. Table 1 summarizes the directions in which each major structure (e.g., the deep fascia 46, the tunica albuginea 48, and the corporeal trabeculae 58) can be stretched.

TABLE 1

| | Axial | Radial | Azimuthal |
|---|---|---|---|
| Structure | Direction | Direction | Direction |
| Deep fascia | Yes | No | Yes |
| Tunica albuginea | Yes | No | Yes |
| Corporeal Trabeculae | Yes | Yes | Yes |

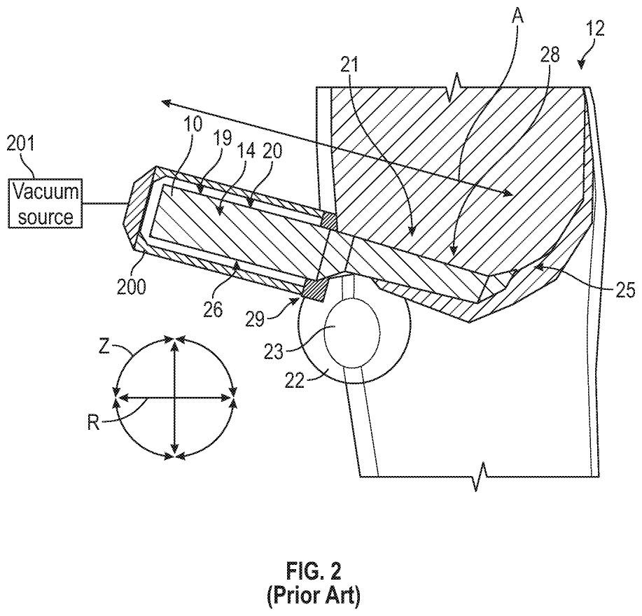

FIG. 2 is a conceptual representation of a schematic, cutaway side view of a user 12 in an area of the penis 10, according to the present disclosure. As shown in FIG. 2, the proximal corpora 28 are attached to a pelvic floor 25 of the user 12. FIG. 2 shows an existing vacuum erection device (VED) 200 applied on the penis 10. The VED 200 is a generally cylindrical vacuum manifold that is sized and shaped to receive the penis 10. For example, the VED 200 is applied to the distal portion 19 of the penile shaft 20. The VED 200 is pressurized by a vacuum source 201 (shown schematically in FIG. 2). In this way, the VED 200 generates a negative pressure about the distal portion 19 of the penile shaft 20 of the penis 10. The VED 200 can generate a negative pressure between 15,000-30,000 Pascals (15-30 kPA). The negative pressure generated by the VED 200 substantially increase (e.g., by approximately double or more) the intracorporeal pressure in the distal portion 19, thereby stretching the tissues of the distal portion 19 of the penis 10 in all three cylindrical dimensions (e.g., in the axial direction A, in the radial direction R, and in the azimuthal direction Z). The proximal corpora 28, however, are left out of the VED 200 and remain unpressurized due to other tissues (e.g., skin from the pubic symphysis and the scrotum 22) being suctioned into an open base of the VED 200, thereby generating an annular “plug” of flesh 29 around the base of the penis 10. The annular plug of flesh 29 seals the vacuum in an area about halfway along the length of the penile corpora 14. The VED 200 transmits axial tension from the distal corpora 26 through the proximal corpora 28 due to the distal corpora 26 being anchored or connected to the body through the proximal corpora 28. The proximal corpora 28, however, remain unpressurized.

FIG. 2 shows the distal corpora 26 inside the VED 200 being stretched width-wise (e.g., along the radial direction R and along the azimuthal direction Z), as well as being stretched length-wise (e.g., along the axial direction A). The proximal corpora 28 are stretched only length-wise (e.g., along the axial direction A), due to being unpressurized by the VED 200. Therefore, the VED 200 does not stretch the proximal corpora 28 width-wise (e.g., along the radial direction R or the azimuthal direction Z). Accordingly, FIG. 2 shows the distal corpora 26 have a larger diameter than the proximal corpora 28 due to the distal corpora 26 being stretched radially and azimuthally, and the proximal corpora 28 being stretched only axially (e.g., not being stretched radially or azimuthally). Table 2 summarizes the stretching dimensions achieved by the VED 200.

TABLE 2

| | Axial | Radial | Azimuthal |
|---|---|---|---|
| Structure | Direction | Direction | Direction |
| Distal Corpora | Yes | Yes | Yes |
| Proximal Corpora | Yes | No | No |

While the VED 200 applies axial stress (e.g., axial stretching) to the proximal corpora 28, enhanced sexual stimulation or treatment of a proximal fibrotic stenosis benefits from applying radial traction (e.g., radial stretching) to the proximal portion of the corporeal trabeculae 58 (FIG. 1D), and/or applying azimuthal traction (e.g., azimuthal stretching) to the proximal portion of the tunica albuginea 48 (FIG. 1D) and to the proximal portion of the deep fascia 46 (FIG. 1D). Accordingly, the present disclosure provides a vacuum manifold for pressurizing the proximal corpora 28 in order to stretch the proximal corpora 28 in the radial direction R and in the azimuthal direction Z, as detailed further below.

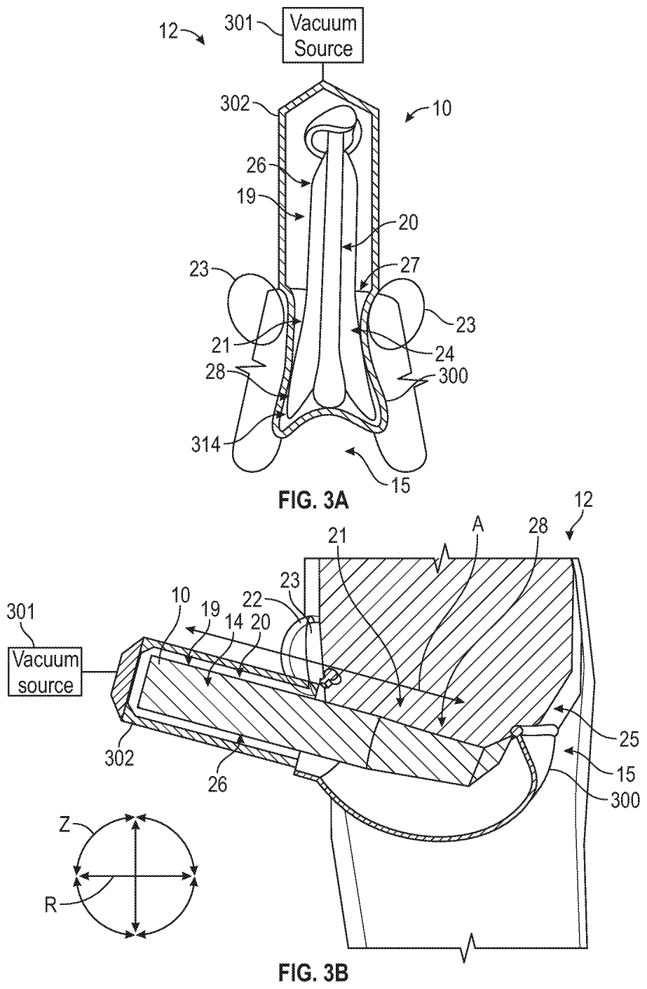

FIG. 3A is a bottom, inferior view of a penis 10 of a user 12 with a vacuum manifold 300 (also referred to as a manifold extension) applied to the user 12, according to the present disclosure. FIG. 3B is a schematic, cutaway side view of the user 12 in an area of the penis 10 with the vacuum manifold 300 applied to the user 12, according to the present disclosure. FIGS. 3A and 3B show a vacuum erection device (VED) 302 applied to distal portion 19 of the penile shaft 20, similar to the VED 200 of FIG. 2. The distal portion 19 of the penile shaft 20 is inserted into the VED 302 and the VED 302 extends to the pubic symphysis 27. Accordingly, the VED 302 applies pressure only to the distal corpora 26. The vacuum manifold 300, also referred to as a manifold extension can be coupled to the VED 302. The vacuum manifold 300 receives the perineum 24 within an inner chamber 314 of the vacuum manifold 300, as detailed further below. The vacuum manifold 300 is sized and is shaped to receive the perineum 24 while excluding the testicles 23 and an anus 15 of the user 12. For example, the scrotum 22, and therefore the testicles 23, are cradled outside of the vacuum manifold 300 when the vacuum manifold 300 is applied to the perineum 24. The vacuum manifold 300 is fluidly coupled to the VED 302 and the assembly is sealed. In use, a vacuum source 301 pressurizes the VED 302, thereby pressurizing the vacuum manifold 300. The vacuum source 301 can include any type of vacuum source for pressurizing the vacuum manifold 300, such as, for example, a manual pump (as illustrated in FIGS. 5 and 7) or an electrical/battery powered pump. The pressurization in the VED 302 stretches the distal corpora 26, as detailed above, thereby stretching the proximal corpora 28 in the axial direction A. The pressurization in the vacuum manifold 300 stretches the proximal corpora 28 in the axial direction A, in the radial direction R, and in the azimuthal direction Z. For example, the vacuum manifold 300 can be used to stretch the corpus cavernosum 54 (FIG. 1D). The vacuum manifold 300 can also be used to stretch the corpus spongiosum 52 (FIG. 1D), thereby assisting with clearing urethral stricture. Thus, the vacuum manifold 300 can be used to help alleviate a proximal fibrotic stenosis, and other ailments of the proximal corpora 28 that currently cannot be fully stretched with current VEDs. Additional features and functions of the vacuum manifold are detailed further below.

FIG. 4 is a perspective view of the vacuum manifold 300 isolated from the VED 302 of FIG. 3, according to the present disclosure. The vacuum manifold 300 includes a body 304 that extends from a proximal end 306 to a distal end 308. The proximal end 306 is also referred to as a posterior end and the distal end is also referred to as an anterior end. The body 304 also extends from a top end 310 to a bottom end 312 that is opposite the top end 310. The body 304 defines an inner chamber 314. The inner chamber 314 is a hollow interior of the body 304. The vacuum manifold 300 also includes a first opening 316 and a second opening 318. The first opening 316 is configured to receive the perineum of the user into the inner chamber 314. The first opening 316 is located at the top end 310 of the body 304 and is elongate from the proximal end 306 to the distal end 308. The first opening 316 is sized such that the testicles of the user rest outside of the inner chamber 314 when the inner chamber 314 receives the perineum. The distal end 308 of the first opening 316 is shaped to receive a pubic area (e.g., a pubic symphysis) of the user. The second opening 318 is configured to receive the penile shaft of the user. The second opening 318 extends from the inner chamber 314 to the distal end 308. The second opening 318 is sized and is shaped to receive the penile shaft. The second opening 318 extends from the distal end 308 of the inner chamber 314 and defines an extension portion 320 of the body 304. The extension portion 320 extends axially from the distal end 308 of the body 304. When the vacuum manifold 300 receives the perineum, the vacuum manifold 300 is configured to form a vacuum generated seal against the perineum of the user, as detailed further below.

The body 304 defines a generally bulbous shape that extends from the first opening 316 to the bottom end 312. In this way, the body 304 is round or is bulging such that the body 304 curves outward from the top end 310 (e.g., from the first opening 316) to the bottom end 312. For example, the body 304 includes a generally semi-spherical shape. The vacuum manifold 300 includes an indented portion 322 at the proximal end 306. For example, the indented portion 322 is defined in the body 304 and forms a part of the shape of the body 304 and of the first opening 316. The indented portion 322 bifurcates the proximal end 306 into two branches 324. For example, indented portion 322 splits the proximal end 306 of the body 304 into a first branch 324a and a second branch 324b. In this way, the indented portion 322 excludes the anus of the user when the inner chamber 314 receives the perineum. FIG. 3A shows the indented portion 322 is shaped to exclude the anus 15 of the user 12. For example, the anus 15 of the user 12 is not disposed in the inner chamber 314 when the vacuum manifold 300 is applied to the perineum 24.

With continued reference to FIG. 4, the vacuum manifold 300 also includes a vacuum attachment 326 configured to provide fluid communication from the vacuum source 301 (FIGS. 3A and 3B) to the inner chamber 314. In this way, the vacuum source 301 provides a vacuum inside the inner chamber 314 of the vacuum manifold 300 to form a vacuum generated seal against the perineum and to pressurize the perineum. The vacuum attachment 326 is the second opening 318 for coupling the vacuum manifold 300 to the VED 302 (FIGS. 3A and 3B), as detailed further below. For example, the extension portion 320 is configured to engage the VED 302 to attach the vacuum manifold 300 to the VED 302.

The vacuum manifold 300 further includes a first raised lip portion 328 on the body 304 that extends about the first opening 316. The first raised lip portion 328 provides a cushion about the body 304 that contacts the user's skin to provide the vacuum generated seal against the perineum of the user.

FIG. 5 is a perspective side view of a system 500 for stretching proximal corpora of a user including the vacuum manifold 300 attached to the VED 302, according to the present disclosure. The vacuum manifold 300 is on a side in FIG. 5 such that the first opening 316 is not facing up. For example, the top end 310 is on the bottom and the bottom end 312 is on the top in FIG. 5. As shown in FIG. 5, the extension portion 320 is sized and is shaped to receive a portion of the VED 302. For example, the VED 302 can be inserted into the extension portion 320. In some embodiments, the extension portion 320 is sized and is shaped such that the extension portion 320 is inserted into the VED 302 to attach the vacuum manifold 300 to the VED 302. The vacuum manifold 300 can be attached to VED 302 by a threaded connection, by a press fit, or by any other connection means, as desired. In some embodiments, the vacuum manifold 300 is formed integrally with the VED 302 such that the vacuum manifold 300 and the VED 302 form a single, unitary component. Accordingly, the vacuum manifold 300 extends from the VED 302.

The system 500 also includes the vacuum source 301. In FIG. 5, the vacuum source 301 is a pump device 550. The pump device 550 includes a pump 552 and a hose 554. The hose 554 is coupled to the VED 302 to provide fluid communication from the pump 552 to the VED 302. In this way, the pump device 550 is in fluid communication with the VED 302 and the vacuum manifold 300 is in fluid communication with the VED 302. The pump 552 as shown in FIG. 5 is a hand pump that can be manually actuated by a user. In addition to the illustrated embodiment, the pump 552 can include any type of pump and can be an electric pump or the like. In use, the user inserts the penis into the first opening 316 and through the inside of the VED 302. When the penis is disposed in the VED 302, the perineum is disposed within the inner chamber 314. The user can then actuate the pump 552 to pressurize the inside of the VED 302, thereby pressurizing the inner chamber 314 of the vacuum manifold 300. In this way, the pressure stretches the penis inside the VED 302, and at the same time, stretches the proximal corpora axially, radially, and azimuthally inside the vacuum manifold 300.

FIG. 6 is a perspective view of a part of a vacuum manifold 600, according to another embodiment. FIG. 7 is a perspective side view of the vacuum manifold 600 attached to a vacuum source 701, according to the present disclosure. With reference to FIGS. 6 and 7, the vacuum manifold 600 includes a body 604 that extends from a proximal end 606 to a distal end 608. The body 604 also extends from a top end 610 to a bottom end 612 that is opposite the top end 610. FIG. 6 shows a first longitudinal hemisphere 611 of the body 604. FIG. 7 shows the first longitudinal hemisphere 611 and a second longitudinal hemisphere 613 of the body. The first longitudinal hemisphere 611 and the second longitudinal hemisphere 613 are defined as a respective half of the body 604 that is divided along a plane (e.g., a mid-sagittal plane) extending along the axial direction of the body 604.

The body 604 defines an inner chamber 614. The inner chamber 614 is a hollow interior of the body 604. The vacuum manifold 600 also includes a first opening 616 and a second opening 618. The first opening 616 is configured to receive the perineum of the user into the inner chamber 614. The first opening 616 is located at the top end 610 of the body 604 and is elongate from the proximal end 606 to the distal end 608. The first opening 616 is sized such that the testicles of the user rest outside of the inner chamber 614 when the inner chamber 614 receives the perineum. The distal end 608 of the first opening 616 is shaped to receive a pubic area (e.g., a pubic symphysis) of the user. The second opening 618 is configured to receive the penile shaft of the user. The second opening 618 extends from the inner chamber 614 to the distal end 608. The second opening 618 is sized and is shaped to receive the penile shaft. The second opening 618 extends from the distal end 608 of the inner chamber 614 and defines an extension portion 620 of the body 604. The extension portion 620 extends axially from the distal end 608 of the body 604. The extension portion 620 is configured to prevent scrotal skin of the user from entering the inner chamber 614 when the inner chamber 614 receives the perineum. When the vacuum manifold 600 receives the perineum, the vacuum manifold 600 is configured to form a vacuum generated seal against the perineum of the user, as detailed further below.

The body 604 defines a generally bulbous shape that extends from the first opening 616 to the bottom end 612. In this way, the body 604 is round or is bulging such that the body 604 curves outward from the top end 610 (e.g., from the first opening 616) to the bottom end 612. For example, the body 604 includes a generally semi-spherical shape. The vacuum manifold 600 includes an indented portion 622 at the proximal end 606. For example, the indented portion 622 is defined in the body 604 and forms a part of the shape of the body 604 and of the first opening 616. The indented portion 622 bifurcates the proximal end 606 into two branches 624. For example, indented portion 622 splits the proximal end 606 of the body 604 into a first branch 624a and a second branch 624b (FIG. 7). In this way, the indented portion 622 excludes the anus of the user 12 when the inner chamber 614 receives the perineum. For example, the anus of the user 12 is not disposed in the inner chamber 614 when the vacuum manifold 600 is applied to the perineum.

The body 604 also includes one or more wings 625 that extend laterally from the body 604. The one or more wings 625 are positioned at the distal end 608 of the body 604 and are configured to secure the testicles of the user outside of the inner chamber 614 when the inner chamber 614 receives the perineum. The one or more wings 625 are sized and are shaped to receive and to secure the testicles. The one or more wings 625 include a first wing 625a and a second wing 625b. The first wing 625a extends laterally from the first longitudinal hemisphere 611 of the body 604. The second wing 625b extends laterally from the second longitudinal hemisphere 613 of the body 604.

The vacuum manifold 600 also includes a vacuum attachment 626 configured to provide fluid communication from a vacuum source 701 (FIG. 7) to the inner chamber 614. In this way, the vacuum source 701 provides a vacuum inside the inner chamber 614 of the vacuum manifold 600 to form a vacuum generated seal against the perineum and to pressurize the perineum. The vacuum attachment 626 is a hose barb 627 that receives a hose 754 (FIG. 7) of the vacuum source 701, as detailed further below.

The vacuum manifold 600 further includes a first raised lip portion 628 and a second raised lip portion 630 on the body 604. The first raised lip portion 628 extends about the first opening 616. The first raised lip portion 628 provides a cushion about the body 604 that contacts the user's skin to provide the vacuum generated seal against the perineum of the user. The second raised lip portion 630 extends about the second opening 618 at the extension portion 620. In this way, a seal is generated when the user's penis is inserted into the second opening 618.

FIG. 7 is a perspective side view of a system 700 for stretching proximal corpora of a user including the vacuum manifold 600 attached to the vacuum source 701, according to the present disclosure. As shown in FIG. 7, the vacuum source 701 is a pump device 750. The pump device 750 includes a pump 752 and a hose 754. The hose 754 is coupled to the hose barb 627 of the vacuum manifold 600 to provide fluid communication from the pump 752 to the vacuum manifold 600. The pump 752 is a hand pump that can be manually actuated by a user. The pump 752 can include any type of pump and can be an electric pump or the like. In use, the user inserts the penis into the first opening 616 and the penile shaft rests on the extension portion 620. When the penis is disposed on the extension portion 620, the perineum is disposed within the inner chamber 614. The user can then actuate the pump 752 to pressurize the inner chamber 614 of the vacuum manifold 600. In this way, the pressure stretches the proximal corpora axially, radially, and azimuthally inside the vacuum manifold 600.

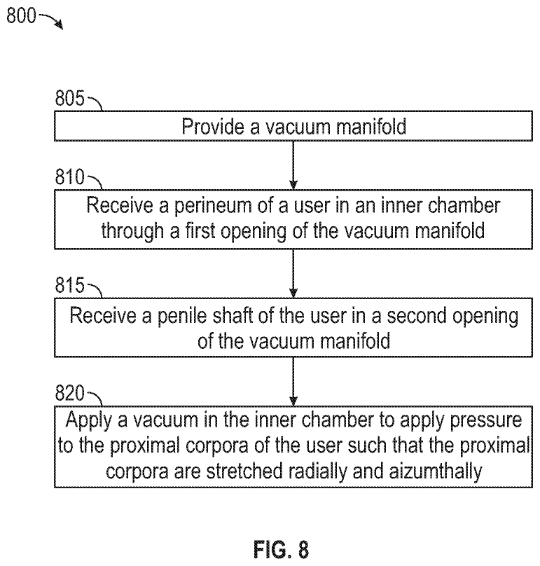

FIG. 8 is a flow diagram of a method 800 of stretching proximal corpora of a user. In step 805, the method 800 includes providing a vacuum manifold. The manifold can include any of the vacuum manifolds disclosed herein. For example, the vacuum manifold includes a body, an inner chamber defined by the body, a first opening in a top end of the body, and a second opening extending from the inner chamber to a distal end of the body.

In step 810, the method 800 includes receiving a perineum of the user in the inner chamber. For example, the perineum is received through the first opening of the body of the vacuum manifold.

In step 815, the method 800 includes receiving a penile shaft of the user in the second opening.

In step 820, the method 800 includes applying a vacuum in the inner chamber to apply pressure to the proximal corpora of the user such that the proximal corpora is stretched radially and azimuthally.

The vacuum manifolds disclosed herein provide for an improved device for stretching the proximal corpora radially and azimuthally. The vacuum manifolds of the present disclosure can be used to ease corporeal fibrosis, as well as for pleasure (e.g., via pressurization of the proximal corpora). Accordingly, the vacuum manifolds and associated methods herein provide for an improved device and methods for stretching the proximal corpora. Table 3 summarizes the stretching dimensions achieved by the vacuum manifolds herein.

TABLE 3

| | Axial | Radial | Azimuthal |
|---|---|---|---|
| Structure | Direction | Direction | Direction |
| Distal Corpora | Yes | Yes | Yes |
| Proximal Corpora | Yes | Yes, improved | Yes, improved |

Further aspects are provided by the subject matter of the following clauses.

A vacuum manifold comprises a body extending from a proximal end to a distal end opposite the proximal end, and from a top end to a bottom end opposite the top end, an inner chamber defined by the body, a vacuum attachment configured to provide fluid communication from a vacuum source to the inner chamber, a first opening for receiving a perineum of a user into the inner chamber, the first opening located at the top end of the body, and a second opening for receiving a penile shaft of the user, the second opening extending from the inner chamber to the distal end, wherein the inner chamber of the vacuum manifold is configured to apply pressure on a proximal corpora of the user such that the proximal corpora is stretched radially and azimuthally.

The vacuum manifold of any preceding clause, wherein the body defines a generally bulbous shape that extends from the first opening to the bottom end.

The vacuum manifold of any preceding clause, further comprising an indented portion at the proximal end.

The vacuum manifold of any preceding clause, wherein the indented portion bifurcates the proximal end into two branches.

The vacuum manifold of any preceding clause, wherein the indented portion is configured to exclude an anus of the user when the inner chamber receives the perineum.

The vacuum manifold of any preceding clause, wherein the vacuum attachment is a hose barb configured to receive a hose of the vacuum source, the hose barb being attached to the body and providing fluid communication from the vacuum source to the inner chamber.

The vacuum manifold of any preceding clause, wherein the vacuum attachment is a vacuum erection device, the vacuum erection device being coupled to the second opening of the vacuum manifold.

The vacuum manifold of any preceding clause, further comprising a first raised lip portion about the first opening, the first raised lip portion providing a vacuum generated seal against the perineum of the user.

The vacuum manifold of any preceding clause, wherein the first opening is elongate from the proximal end to the distal end.

The vacuum manifold of any preceding clause, wherein the first opening is sized such that testicles of the user rest outside of the inner chamber when the inner chamber receives the perineum.

The vacuum manifold of any preceding clause, wherein the distal end of the first opening is shaped to receive a pubic symphysis of the user.

The vacuum manifold of any preceding clause, further comprising one or more wings that extend laterally from the body, the one or more wings positioned at the distal end and configured to secure testicles of the user outside of the inner chamber when the inner chamber receives the perineum.

The vacuum manifold of any preceding clause, wherein the one or more wings include a first wing and a second wing.

The vacuum manifold of any preceding clause, wherein the first wing extends laterally from a first longitudinal hemisphere of the body, and the second wing extends laterally from a second longitudinal hemisphere of the body.

The vacuum manifold of any preceding clause, wherein the second opening extends from the distal end of the inner chamber and defines an extension portion of the body, the extension portion configured to prevent scrotal skin of the user from entering the inner chamber when the inner chamber receives the perineum.

The vacuum manifold of any preceding clause, further including a second raised lip portion about the second opening at the extension portion.

The vacuum manifold of any preceding clause, wherein the vacuum source applies a pressure in the inner chamber for stretching the distal corpora of the user.

The vacuum manifold of any preceding clause, wherein the vacuum source is a pump device.

The vacuum manifold of any preceding clause, wherein the pump device is a hand pump.

The vacuum manifold of any preceding clause, wherein the pump device is an electric pump.

A system for stretching proximal corpora of a user, the system comprises a vacuum erection device for receiving a distal portion of a penile shaft of the user, a vacuum source coupled to the vacuum erection device, and a vacuum manifold extending from the vacuum erection device. The vacuum manifold comprises a body extending from a proximal end to a distal end opposite the proximal end, and from a top end to a bottom end opposite the top end; an inner chamber defined by the body, the inner chamber being in fluid communication with the vacuum erection device, a first opening for receiving a perineum of the user into the inner chamber, the first opening located at the top end of the body, and a second opening for receiving the penile shaft of the user, the second opening extending from the inner chamber to the distal end. The inner chamber of the vacuum manifold is configured to apply pressure from the vacuum source on a proximal corpora of the user such that the proximal corpora is stretched radially and azimuthally.

The system of the preceding clause, wherein the vacuum source applies a pressure in the vacuum erection device for stretching a distal corpora of the user.

The system of any preceding clause, wherein the vacuum source is a pump device.

The system of any preceding clause, wherein the pump device is a hand pump.

The system of any preceding clause, wherein the pump device is an electric pump.

The system of any preceding clause, wherein the vacuum erection device is generally cylindrical and is sized to receive the penile shaft of the user.

The system of any preceding clause, wherein the body defines a generally bulbous shape that extends from the first opening to the bottom end.

The system of any preceding clause, wherein the vacuum manifold further comprises an indented portion at the proximal end.

The system of any preceding clause, wherein the indented portion bifurcates the proximal end into two branches.

The system of any preceding clause, wherein the indented portion is configured to exclude an anus of the user when the inner chamber receives the perineum.

The system of any preceding clause, wherein the vacuum manifold further comprises a first raised lip portion about the first opening, the first raised lip portion providing a vacuum generated seal against the perineum of the user.

The system of any preceding clause, wherein the first opening is elongate from the proximal end to the distal end.

The system of any preceding clause, wherein the first opening is sized such that testicles of the user rest outside of the inner chamber when the inner chamber receives the perineum.

The system of any preceding clause, wherein the distal end of the first opening is shaped to receive a pubic symphysis of the user.

The system of any preceding clause, wherein the vacuum manifold further comprises one or more wings that extend laterally from the body, the one or more wings positioned at the distal end and configured to secure testicles of the user outside of the inner chamber when the inner chamber receives the perineum.

The system of any preceding clause, wherein the one or more wings include a first wing and a second wing.

The system of any preceding clause, wherein the first wing extends laterally from a first longitudinal hemisphere of the body, and the second wing extends laterally from a second longitudinal hemisphere of the body.

The system of any preceding clause, wherein the second opening extends from the distal end of the inner chamber and defines an extension portion of the body, the extension portion configured to prevent scrotal skin of the user from entering the inner chamber when the inner chamber receives the perineum.

The system of any preceding clause, further including a second raised lip portion about the second opening at the extension portion.

A method of stretching proximal corpora of a user comprises providing a vacuum manifold having a body, an inner chamber defined by the body, a first opening in a top end of the body, and a second opening extending from the inner chamber to a distal end of the body, receiving a perineum of the user in the inner chamber, receiving a penile shaft of the user in the second opening, and applying a vacuum in the inner chamber to apply pressure to the proximal corpora of the user such that the proximal corpora are stretched radially and azimuthally.

The method of any preceding clause, further comprising providing an indented portion in the body, and separating an anus of the user from the inner chamber by the indented portion when the perineum is received in the inner chamber.

The method of any preceding clause, wherein applying the vacuum includes generating the vacuum with a vacuum source that is in fluid communication with the inner chamber.

The method of any preceding clause, wherein the body includes a hose barb, and the method further comprises providing fluid communication from the vacuum source to the inner chamber through the hose barb.

The method of any preceding clause, further comprising providing a vacuum erection device coupled to the body at the second opening, and the method further comprises providing fluid communication from the vacuum source to the inner chamber through the vacuum erection device.

The method of any preceding clause, further comprising providing a first raised lip portion about the first opening, and generating a vacuum seal against the perineum with the first raised lip portion.

The method of any preceding clause, wherein the first opening is sized such that testes of the user rest outside of the inner chamber when the inner chamber receives the perineum.

The method of any preceding clause, wherein the distal end of the first opening is shaped to receive a pubic symphysis of the user when the inner chamber receives the perineum.

The method of any preceding clause, further comprising providing one or more wings that extend laterally from the body, and securing testicles of the user by the one or more wings when the inner chamber receives the perineum.

The method of any preceding clause, wherein the one or more wings include a first wing and a second wing.

The method of any preceding clause, wherein the first wing extends laterally from a first longitudinal hemisphere of the body, and the second wing extends laterally from a second longitudinal hemisphere of the body.

The method of any preceding clause, further comprising providing an extension portion at the second opening, and preventing scrotal skin of the user from entering the inner chamber by the extension portion when the inner chamber receives the perineum.

The method of any preceding clause, further comprising providing a second raised lip portion about the second opening at the extension portion.

The method of any preceding clause, wherein the vacuum source is a pump device, and the method further comprises generating the pressure in the inner chamber with the pump device.

The method of any preceding clause, wherein the pump device is a hand pump.

The method of any preceding clause, wherein the pump device is an electric pump.

Although the foregoing description is directed to the preferred embodiments of the present disclosure, other variations and modifications will be apparent to those skilled in the art and may be made without departing from the spirit or the scope of the disclosure. Moreover, features described in connection with one embodiment of the present disclosure may be used in conjunction with other embodiments, even if not explicitly stated above.

1. A vacuum manifold comprising:
a body extending from a proximal end to a distal end opposite the proximal end, and from a top end to a bottom end opposite the top end;
an inner chamber defined by the body;
a vacuum attachment configured to provide fluid communication from a vacuum source to the inner chamber;
a first opening for receiving a perineum of a user into the inner chamber, the first opening located at the top end of the body; and
a second opening for receiving a penile shaft of the user, the second opening extending from the inner chamber to the distal end,
wherein the inner chamber of the vacuum manifold is configured to apply pressure on a proximal corpora of the user such that the proximal corpora is stretched radially and azimuthally.

2. The vacuum manifold of claim 1, wherein the body defines a generally bulbous shape that extends from the first opening to the bottom end.

3. The vacuum manifold of claim 1, further comprising an indented portion at the proximal end.

4. The vacuum manifold of claim 3, wherein the indented portion bifurcates the proximal end into two branches.

5. The vacuum manifold of claim 3, wherein the indented portion is configured to exclude an anus of the user when the inner chamber receives the perineum.

6. The vacuum manifold of claim 1, wherein the vacuum attachment is a hose barb configured to receive a hose of the vacuum source, the hose barb being attached to the body and providing fluid communication from the vacuum source to the inner chamber.

7. The vacuum manifold of claim 1, wherein the vacuum attachment is a vacuum erection device, the vacuum erection device being coupled to the second opening of the vacuum manifold.

8. The vacuum manifold of claim 1, further comprising a first raised lip portion about the first opening, the first raised lip portion providing a vacuum generated seal against the perineum of the user.

9. The vacuum manifold of claim 1, wherein the first opening is elongate from the proximal end to the distal end.

10. The vacuum manifold of claim 9, wherein the first opening is sized such that testicles of the user rest outside of the inner chamber when the inner chamber receives the perineum.

11. The vacuum manifold of claim 9, wherein the distal end of the first opening is shaped to receive a pubic symphysis of the user.

12. The vacuum manifold of claim 1, further comprising one or more wings that extend laterally from the body, the one or more wings positioned at the distal end and configured to secure testicles of the user outside of the inner chamber when the inner chamber receives the perineum.

13. The vacuum manifold of claim 12, wherein the one or more wings include a first wing and a second wing.

14. The vacuum manifold of claim 13, wherein the first wing extends laterally from a first longitudinal hemisphere of the body, and the second wing extends laterally from a second longitudinal hemisphere of the body.

15. The vacuum manifold of claim 1, wherein the second opening extends from the distal end of the inner chamber and defines an extension portion of the body, the extension portion configured to prevent scrotal skin of the user from entering the inner chamber when the inner chamber receives the perineum.

16. The vacuum manifold of claim 15, further including a second raised lip portion about the second opening at the extension portion.

17. A system for stretching proximal corpora of a user, the system comprising:
a vacuum erection device for receiving a distal portion of a penile shaft of the user;
a vacuum source coupled to the vacuum erection device; and
a vacuum manifold extending from the vacuum erection device, the vacuum manifold comprising:
a body extending from a proximal end to a distal end opposite the proximal end, and from a top end to a bottom end opposite the top end;
an inner chamber defined by the body, the inner chamber being in fluid communication with the vacuum erection device;
a first opening for receiving a perineum of the user into the inner chamber, the first opening located at the top end of the body; and
a second opening for receiving the penile shaft of the user, the second opening extending from the inner chamber to the distal end,
wherein the inner chamber of the vacuum manifold is configured to apply pressure from the vacuum source on a proximal corpora of the user such that the proximal corpora is stretched radially and azimuthally.

18. The system of claim 17, wherein the vacuum source applies a pressure in the vacuum erection device for stretching a distal corpora of the user.

19. The system of claim 17, wherein the vacuum erection device is generally cylindrical and is sized to receive the penile shaft of the user.

20. A method of stretching proximal corpora of a user, the method comprising:
providing a vacuum manifold having a body, an inner chamber defined by the body, a first opening in a top end of the body, and a second opening extending from the inner chamber to a distal end of the body;
receiving a perineum of the user in the inner chamber;
receiving a penile shaft of the user in the second opening; and
applying a vacuum in the inner chamber to apply pressure to the proximal corpora of the user such that the proximal corpora are stretched radially and azimuthally.
